(19) United States
(12) Patent Application Publication
(10) Pub. No.: US 2023/0372646 A1
(43) Pub. Date: Nov. 23, 2023

(54) INHALING DEVICE

(71) Applicant: Willie Tsang, Toronto (CA)

(72) Inventor: Willie Tsang, Toronto (CA)

(21) Appl. No.: 17/748,676

(22) Filed: May 19, 2022

(51) Int. Cl. A61M 15/08; A61M 15/00

(52) U.S. Cl. CPC A61M 15/08; A61M 15/0091; A61M 15/002; A61M 2205/586; A61M 2205/3334

(57) ABSTRACT

An inhaling device for attachment to a container is provided. The inhaling device has a first body defining an inhalation vent, an air intake that is in fluid communication with the inhalation vent, a second body configured to attach to an opening of the container, and a valve that opens and closes fluid communication between an external portion of the inhalation vent and the opening of the container in an open position and a closed position respectively. A kit containing the inhaling device and diffusion material is also provided.

### FIELD

The present disclosure relates to inhaling devices, and more specifically inhaling devices for attachment to containers and inhaling their contents.

### BACKGROUND

The statements in this section merely provide background information related to the present disclosure and may not constitute prior art.

Essential oils may be broadly understood as a concentrated liquid containing volatile chemical compounds from natural sources, such as plants. To enjoy their scent, a user may remove the cap from a bottle of essential oil and inhale it directly from the bottle.

Alternatively, a user may use a diffuser or an inhaler. Diffusers typically work by adding several drops of oil to an inner chamber of water to produce scented steam that fills a small room. Inhalers often take the form of disposable wicks or pre-infused cores that emit a scent when exposed to air. A user may put the wick or core at a close proximity to their nose to inhale the emitted scent. The wicks may be pre-infused with essential oil and placed into a housing for passive diffusion into the surrounding environment.

Directly inhaling essential oils may result in irritation caused by direct exposure of essential oils to the user's skin or nose. A bottle with an open cap may also be vulnerable to spills and/or splashing. Diffusers commonly suffer from low portability. Diffusers or inhalers may also leak, spill or drip when knocked over.

There is a need for an inhaling device that addresses one or more of these drawbacks.

### SUMMARY

This section provides a general summary of the disclosure and is not a comprehensive disclosure of its full scope or all of its features.

The present disclosure relates to apparatuses and kits for inhaling a scent from a container, such as a bottle, holding an evaporating liquid. Inhaling devices disclosed herein provide valve means to substantially seal the container from leaking fluid, such as liquid or gas. Such inhaling devices may facilitate the intake of scents, such as aromas, from the container through an inhalation vent. Inhaling devices disclosed herein may be configured to removably fit or seal onto the opening of a container.

According to an aspect of the present disclosure, there is provided an inhaling device for attachment to a container, the inhaling device comprising a first body defining an inhalation vent, an air intake that is in fluid communication with the inhalation vent, a second body configured to attach to an opening of the container, and a valve that opens and closes fluid communication between an external portion of the inhalation vent and the opening of the container in an open position and a closed position, respectively.

In some embodiments, the valve comprises a valve member that is operable to obstruct the inhalation vent in the closed position.

In some embodiments, the first body is movably attached to the second body, and moving the first body actuates the valve between the open position and the closed position. In some embodiments, the first body is movably attached to the second body via a cylinder cam. In some embodiments, the second body comprises the cylinder cam, and the first body comprises a follower that is configured to ride the cylinder cam.

In some embodiments, the first body is twistable around the second body. In further embodiments, the inhaling device is sized for single-handedly twisting while holding the bottle.

In some embodiments, in the open configuration the first body is spaced from the second body along a central axis, and in the closed configuration the first body is proximate the second body along the central axis.

In some embodiments, the first body covers the second body, the air intake is formed in a gap defined by a base of the second body, and the first body is movable between a first position in which the gap is closed and the valve is in the closed position, and a second position in which the gap is open and the valve is in the open position.

In some embodiments, the inhaling device further comprises a channel defined by the second body and providing fluid communication between the inhalation vent and the air intake. In some embodiments, the inhaling device further comprises a plurality of channels inclusive of the channel, each channel of the plurality of channels providing fluid communication between the inhalation vent and the air intake. In some embodiments, the channels are disposed radially around the second body.

In some embodiments, the inhaling device further comprises a diffusion chamber, wherein the inhalation vent is in fluid communication with the air intake via the diffusion chamber.

In some embodiments, the inhaling device further comprises a fastener for sealing to the opening of the container, wherein the container is a bottle, and the diffusion chamber is disposed above the fastener. In some embodiments, the diffusion chamber is formed by walls of the first body and the second body. In some embodiments, the second body comprises a retaining chamber that is configured for retaining a diffusion material and disposed proximate to the diffusion chamber.

In some embodiments, the inhalation vent is shaped to be received in a nostril of a user.

According to another aspect of the present disclosure, there is provided a kit comprising an inhaling device as described above, and diffusing material sized to form a seal a retaining chamber of the inhaling device. In some embodiments, the diffusing material is suitable for diffusing an evaporating liquid from the bottle for inhalation. In some embodiments, the diffusing material is a diffuser wick.

Other aspects and features of the disclosure will become apparent to those ordinarily skilled in the art upon review of the following description of specific embodiments of the disclosure.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

FIG. 1 shows a perspective view of an inhaling device;

FIG. 2 shows a side elevation view of the inhaling device of FIG. 1;

FIG. 2A shows a cross-sectional view taken along line 2A-2A in FIG. 2;

FIG. 3 is a top plan view of the inhaling device of FIG. 1;

FIG. 4 is a bottom plan view of the inhaling device of FIG. 1;

FIG. 5 is a side elevation view of the inhaling device of FIG. 1;

FIG. 6 is a side elevation view of the inhaling device of FIG. 1 secured to a container;

FIG. 7 is a perspective view of a second body of an inhaling device;

FIG. 8 is an exploded view of a first body of an inhaling device and the second body as shown in FIG. 7;

FIG. 9 is a perspective view of the inhaling device of FIG. 8 in the open position;

FIG. 10 is a perspective view of the inhaling device of FIG. 9 in the closed position;

FIG. 10A is a bottom plan view of the inhaling device of FIG. 10; and

FIGS. 11A-11G show steps of a method of installing and using the inhaling device, wherein: FIG. 11A shows a diffuser wick is secured within the inhaling device prior to attachment to a bottle; FIG. 11B shows a bottle lid is removed from a bottle; FIG. 11C shows the inhaling device is secured to an opening of the bottle; FIG. 11D shows a first body and a valve of the inhaling device are moved to an open position; FIG. 11E shows an inhalation vent and an air intake are opened; FIG. 11F shows the inhaling device is placed near a nose of a user; and FIG. 11G shows that the first body and the valve are moved to a closed position.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

One or more illustrative embodiments have been described by way of example. Described herein are inhaler devices for inhaling diffusible liquids, such as essential oils. It will be appreciated that embodiments and examples are provided for illustrative purposes intended for those skilled in the art, and are not meant to be limiting in any way. All references to embodiments, kits, and the like is intended to be illustrative and non-limiting.

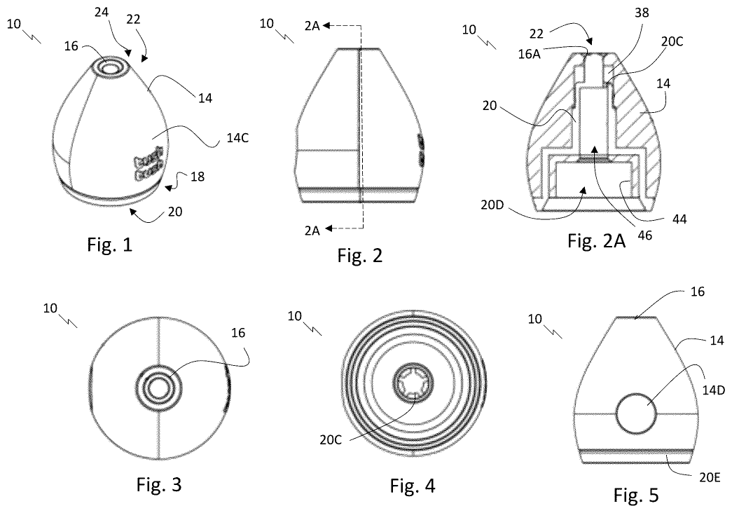

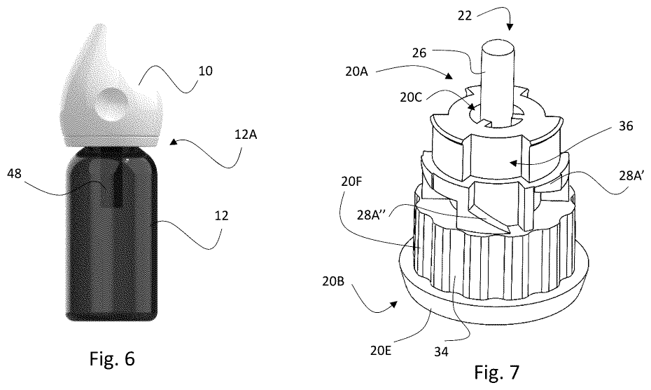

FIGS. 1 and 6, show an inhaling device 10 for attachment to a bottle or container 12. A bottle 12 may house a suitable substance for inhalation, such as an essential oil, fragrance oil, evaporating liquid, and others. Inhaling device 10 comprises a first body 14 defining an inhalation vent 16, an air intake 18 that is in fluid communication with the inhalation vent 16, a second body 20 configured to attach to an opening 12A of the bottle 12, and a valve 22 that opens and closes fluid communication between an external portion 24 of the inhalation vent 16 and opening 12A in an open position and a closed position, respectively.

Referring to FIGS. 2A, 7, and 8, the inhaling device 10 comprises the valve 22 with an open position and a closed position. The valve 22 may prevent fluids, such as air, diffused aromatic molecules or liquids, from leaking or spilling from the bottle 12 and/or the inhaling device 10, when in use and in the closed position. The open position of the valve 22 permits fluid communication between the inhalation vent 16 and the air intake 18. The valve 22, in the open position, may also permit the intake of air through the air intake 18 and air to vent through the inhalation vent 16. The valve 22 may comprise a valve member 26 that is operable to obstruct the inhalation vent 16 in the closed position. The valve member 26 may co-operate with a perimeter 16A of the inhalation vent 16 to substantially seal or close inhalation vent when in the closed position. In some cases, the valve member 26 is an elongate body extending from the second body 20. Other embodiments of the valve member 26 that seal against the inhalation vent 16 are considered, such as a ring, gasket, and others.

Referring to FIGS. 1-5 and 8, the first body 14 may form a protective housing around the second body 20. First body 14 defines inhalation vent 16, which may be at or near a top end 14A of the first body 14. As shown in FIG. 1, the first body 14 may at least partially house the second body 20. The first body 14 has a suitable shape, for example a teardrop shape. An exterior surface 14C of the first body 14 may define a recessed area or gripping portion 14D shaped to receive a finger of a user. In some cases, the gripping portion 14D is a plurality of portions spaced along the exterior surface 14C.

Referring to FIGS. 1 and 2A, the inhalation vent 16 may facilitate the flow of air from the contents of the bottle 12 to a nostril of a user. The inhalation vent 16 may have a suitable shape, for example a shape to be received in a nostril of a user.

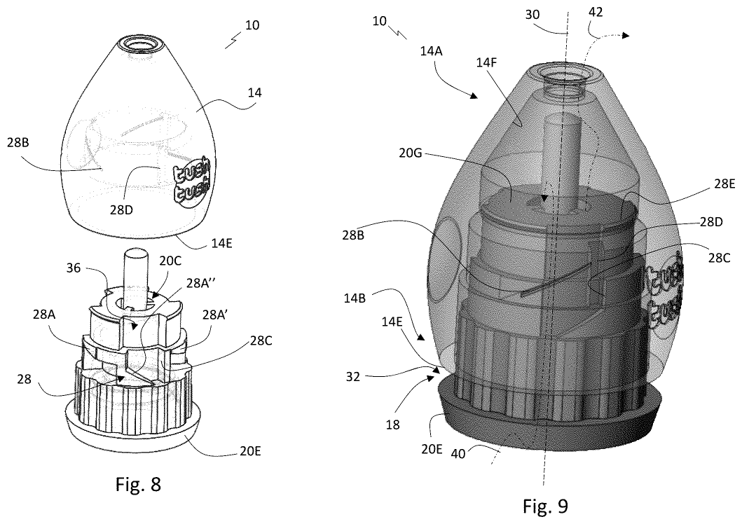

Referring to FIGS. 7 and 8, the second body 20 may nest substantially within the first body 14. The second body 20 may comprise a first end 20A near a top of the second body 20 comprising the valve 22. The first end or portion 20A may also define one or more diffusion vents 20C in fluid communication with the opening 12A of the bottle 12.

In use, diffused aromatic compounds and/or airflow from an interior of the bottle 12 may pass through the opening 12A into an interior 20D of the second body 20. The diffused compounds may then pass-through the diffusion vents 20C to the inhalation vent 16. If the valve 22 is in the open position, the air flow and/or diffused compounds may pass through the inhalation vents into the exterior portion 24 of the inhalation vent 16. In some cases, the diffused compounds and/or airflow may pass through the vent 16 directly into the nostril of the user. If the valve 22 is in the closed position, the air flow and/or diffused compounds may not flow or pass-through vent 16.

Referring to FIG. 8, the first body 14 may be movably attached to second body 20. The first body 14 may be attached to the second body 20 such that the first body 14 moves independently of the second body 20. In some cases, the second body 20 is secured to the opening 12A and the first body 14 moves independently of the second body 20 and the bottle 12. Movement of the first body 14 may actuate the valve 22 between the open position and the closed position.

The first body 14 may move from a first position, where the first body 14 is proximal or adjacent to the second body 20, to a second position, where the first body 14 is spaced from the second body 20. In such cases, the first position may be defined as a closed position in which the first body 14 is in close proximity to the second body 20 to close the valve 22 (FIG. 1). For example, the first position of the first body 14 may be a position in which the valve member 26 is engaging the perimeter 16A of the vent 16 such that the valve 22 is in the closed position. In some cases, the first/closed position is defined as the first body 14 being proximate to the second body 20 along a central axis 30.

The second position of the first body 14 may be defined as an open position in which the first body 14 is spaced from the second body 20 such that the valve 22 is in the open position (FIG. 9). For example, the second position of the first body 14 may be defined as a position in which the valve member 26 is spaced from the perimeter 16A of the vent 16 such that airflow is permitted through the vent 16. In some cases, the second/open position of the first body is defined as the first body 14 being spaced from the second body 20 along the central axis 30.

Referring to FIG. 8, the first body 14 may be movably attached to the second body 20 via a suitable mechanism. For example, the movable connection may be a cam, such as a cylinder cam 28. The cylinder cam 28 may be used to convert a twisting or rotational force to a translating motion, such as translating the first body 14 between the first and second positions. In such cases, twisting the first body 14 may translate or move the first body 14 relative to the second body 20. The cylinder cam 28 may comprise one or more cam walls 28A and the device 10 may comprise one or more followers 28B. One or more followers 28B may be positioned and shaped to ride on corresponding one or more cam walls 28A such that movement of the followers 28B along the walls 28A translates or moves the first body 14 relative to the second body 20. In some embodiments, the cylinder cam 28, such as the walls 28A, are formed or mounted on the second body 20. In some cases, the one or more cam walls 28A comprise upper cam walls 28A′ and lower cam walls 28A″. The cylinder cam 28 may also comprise a stop 28C positioned to abut against a member 28D to prevent further movement of the first body in the open position. Second body 20 may also comprise upper rim 28E to abut against a portion of first body 14 to prevent further movement. The member 28D may also act as a follower 28B positioned to ride along the wall 28A, such as lower wall 28A″.

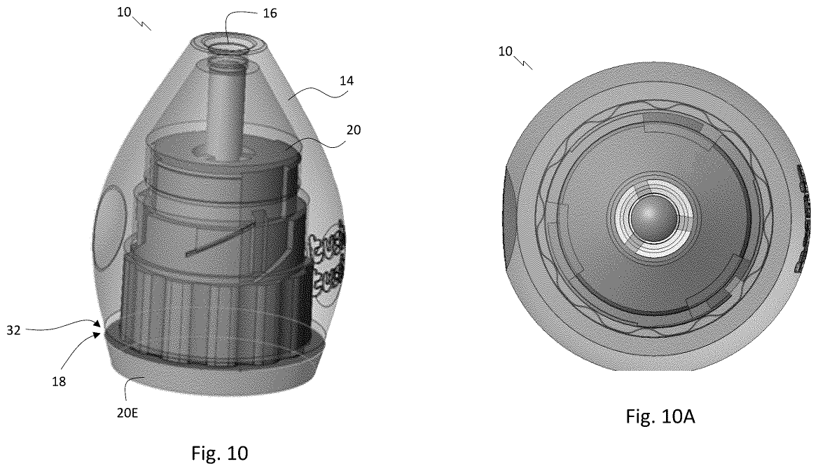

Referring to FIGS. 9, 10, and 10A, the inhaling device 10 comprises the air intake 18 in fluid communication with the inhalation vent 16. The air intake 18 may act to replenish air that is consumed in use by a user. For example, when a user inhales air through the inhalation vent 16, air may evacuate the inner space of bottle 12 (for example via a flow path 42) and may create a vacuum within bottle 12. The air intake 18 may replenish the air that is evacuated from the bottle 12 during use (for example via flow path 40), and provide a more continuous use of the inhaling device 10.

The air intake 18 may be a gap 32 formed by a rim 14E at a bottom end 14B of the first body 14 and a top surface of a base 20E of the second body 20. When the first body 14 and the valve 22 are in the closed position, the gap 32 may be closed as well. While in the closed position, airflow may not be permitted through the gap 32. When the first body 14 and the valve 22 are in the open position, the gap 32 may be open and airflow permitted through the gap 32 and into the inhalation vent 16.

Referring to FIGS. 7-9, the second body 20 may define a channel 34 providing fluid communication between the inhalation vent 16 and the air intake 18. The channel 34 may provide a path for airflow travelling from the air intake 18 along an outer surface 20F of the second body 20 to the inhalation vent 16. The channel 34 may be disposed radially and run along a length of the second body 20. In some cases, the channel 34 is a plurality of channels 34, each channel of the plurality of channels providing fluid communication between the inhalation vent 16 and the air intake 18. The plurality of channels may be disposed radially around the second body 20. In some cases, the channel 34 connects with another channel or structure, such as a passageway 36. The passageway 36 may provide additional flow across the outer surface 20F.

Referring to FIG. 9, the inhaling device 10 may have a diffusion chamber 38 in fluid communication with the air intake 18 and the inhalation vent 16. The diffusion chamber 38 may provide a space where air from the interior of the bottle, for example via the flow path 42, and air from the exterior of the bottle, for example via the flow path 40, can mix before exiting the inhalation vent 16 in use. The diffusion chamber 38 may be formed by walls of the first body 14 and the second body 20, for example an inner wall 14F and a top wall 20G, respectively. The diffusion chamber 38 may be disposed such that flow from both the interior of the bottle 12 and the air intake 18 can combine, such as above the fastener 44.

Referring to FIGS. 2A and 6, the inhaling device 10 may have a retaining chamber 46 configured for retaining a diffusion material. A diffusion material may refer to a structure of material, such as a diffuser wick, that can absorb and diffuse aromatic compounds. The structure may be an elongated body, such as a cylinder 48. Examples of diffusive materials are known in the art, such as cotton, and others. The retaining chamber 46 may be disposed proximate to the diffusion vents 20C and/or the diffusion chamber 38. The retaining chamber 46 and diffusion material may be disposed such that the diffusion material extends into the interior of the bottle 12. The diffusion material may absorb the contents of the bottle, such as an evaporating liquid or essential oil, and provide diffusive properties to diffuse the oil more readily into the surrounding air. In some cases, the diffused oil will travel through the diffusion vents 20C into diffusion chamber 38 and into an exterior of the inhaling device 10 via the inhalation vent 16. The diffusion material may retain the contents of the bottle to prevent movement of the retained liquid when the bottle 12 and the inhaling device 10 are moved unexpectedly. Retaining liquid within the diffusion material may contribute to the prevention of spilling, leaking or splashing of the contents of bottle 12. The diffusion material 48 may extend to or near a bottom of the interior of bottle 12. In some cases, the diffusion material 48 extends to an upper portion of the bottle. The retaining chamber 46 may define retaining members (not shown) such as grooves or ribs to grip the diffusion material. In some cases, the retaining chamber 46 is one of a plurality of retaining chambers for retaining a plurality of diffuser wicks of diffusion material.

Referring to FIG. 2A, the inhaling device 10 may comprise a fastener 44 for releasably attaching and/or sealing to the opening of the bottle 12. The fastener 44 may be removably attachable to the opening 12A such that the device 10 may be detached from the bottle when desired, such as when a different scent is desired or the bottle is empty. The fastener 44 may have a suitable fastening mechanism, such as pin or box threading, that is configured to secure to the opening 12A, such as box or pin threading, respectively. The fastening mechanism may be a suitable mechanism known in the art, such as a friction fit, sealing member such as a gasket, and others. The fastener 44 may be disposed at or near the second end 20B of the second body 20.

The fastener 44 may attach to the bottle 12, which may have a rigid construction. In some cases, the bottle 12 is a B or C bottle. In a specific example, the fastener 44 and the inhaling device 10 are sized to fasten to bottles that have a volume of 1 to 8 ounces or greater.

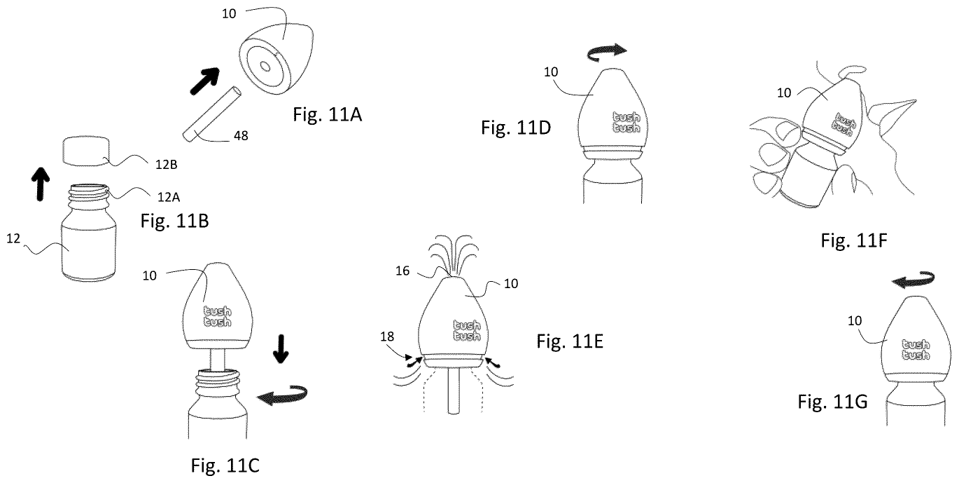

FIGS. 11A-11G show a method of installing and using the inhaling device 10. As shown in FIG. 11A, a diffuser wick 48 may be secured within the inhaling device 10 prior to attachment to the bottle 12. A conventional bottle lid 12B may then be removed from the opening 12A (FIG. 11B). The inhaling device 10 may be secured to the opening 12A of the bottle 12 (FIG. 11C). In cases where a diffusion material is retained within retaining chamber, the diffusion material may extend into the interior of the bottle and at least partially absorb some of the contents therein. The first body 14 and the valve 22 may be initially in the closed position, preventing fluid from entering or exiting the inhalation vent 16 and the air intake 18. A user may then operate the first body 14, such as by twisting, to move the first body 14 and the valve 22 to the open position and open the inhalation vent 16 and the air intake 18 (FIGS. 11D and 11E). The inhaling device 10 may be sized such that a user may single-handedly twist the first body 14 while holding the bottle 12. The user may then place the inhalation vent 16 at or near their nose and inhale the diffused contents of the bottle 12 (FIG. 11F). The user may then move the first body 14 and the valve 22 to the closed position, such as by twisting the first body 14 in a reverse direction around the second body 20 (FIG. 11G). The inhaling device 10 may then be removed from the bottle 12 or remain attached.

Disclosed herein is a kit comprising: the inhaling device 10, and the diffusing material sized to form a seal in the retaining chamber 46.

What has been described is merely illustrative of the application of the principles of the disclosure. However, it will be apparent to a person skilled in the art that a number of variations and modifications can be made without departing from the scope of the following claims.

Unless otherwise expressly indicated herein, all numerical values indicating mechanical/thermal properties, compositional percentages, dimensions and/or tolerances, or other characteristics are to be understood as modified by the word “about” or “approximately” in describing the scope of the present disclosure. This modification is desired for various reasons including industrial practice, material, manufacturing, and assembly tolerances, and testing capability.

As used herein, the phrase at least one of A, B, and C should be construed to mean a logical (A OR B OR C), using a non-exclusive logical OR, and should not be construed to mean “at least one of A, at least one of B, and at least one of C.”

The description of the disclosure is merely exemplary in nature and, thus, variations that do not depart from the substance of the disclosure are intended to be within the scope of the disclosure. Such variations are not to be regarded as a departure from the spirit and scope of the disclosure.

What is claimed is:

1. An inhaling device for attachment to a container, the inhaling device comprising:
a first body defining an inhalation vent;
an air intake that is in fluid communication with the inhalation vent;
a second body configured to attach to an opening of the container; and
a valve that opens and closes fluid communication between an external portion of the inhalation vent and the opening of the container in an open position and a closed position respectively.

2. The inhaling device of claim 1, wherein the valve comprises a valve member that is operable to obstruct the inhalation vent in the closed position.

3. The inhaling device of claim 1, wherein the first body is movably attached to the second body, and wherein moving the first body actuates the valve between the open position and the closed position.

4. The inhaling device of claim 3, wherein the first body is movably attached to the second body via a cylinder cam.

5. The inhaling device of claim 4, wherein the second body comprises the cylinder cam, and the first body comprises a follower that is configured to ride the cylinder cam.

6. The inhaling device of claim 3, wherein the first body is twistable around the second body.

7. The inhaling device of claim 6, wherein the inhaling device is sized for single-handedly twisting while holding the container.

8. The inhaling device of claim 3, wherein in the open position the first body is spaced from the second body along a central axis, and in the closed position the first body is proximate the second body along the central axis.

9. The inhaling device of claim 3, wherein the first body covers the second body, the air intake is formed in a gap defined by a base of the second body, and the first body is movable between a first position in which the gap is closed and the valve is in the closed position, and a second position in which the gap is open and the valve is in the open position.

10. The inhaling device of claim 1, further comprising a channel defined by the second body and providing fluid communication between the inhalation vent and the air intake.

11. The inhaling device of claim 10, further comprising a plurality of channels inclusive of the channel, each channel of the plurality of channels providing fluid communication between the inhalation vent and the air intake.

12. The inhaling device of claim 11, wherein the channels are disposed radially around the second body.

13. The inhaling device of claim 1, further comprising a diffusion chamber, wherein the inhalation vent is in fluid communication with the air intake via the diffusion chamber.

14. The inhaling device of claim 13, further comprising a fastener for sealing to the opening of the container, wherein the container is a bottle, and the diffusion chamber is disposed above the fastener.

15. The inhaling device of claim 13, wherein the diffusion chamber is formed by walls of the first body and the second body.

16. The inhaling device of claim 15, wherein the second body comprises a retaining chamber that is configured for retaining a diffusion material and is disposed proximate to the diffusion chamber.

17. The inhaling device of claim 1, wherein the inhalation vent is shaped to be received in a nostril of a user.

18. A kit comprising:
the inhaling device of claim 1, and
diffusing material sized to form a seal in a retaining chamber of the inhaling device.

19. The kit of claim 18, wherein the diffusing material is suitable for diffusing an evaporating liquid from the container for inhalation.

20. The kit of claim 19, wherein the diffusing material is a diffuser wick.
